(19) United States
(12) Patent Application Publication
(10) Pub. No.: US 2023/0040045 A1
(43) Pub. Date: Feb. 9, 2023

(54) Detection of Viral Nucleic Acid and Its Variant Using Nanopore

(71) Applicant: Genvida Technology Company Limited, Tortola (VG)

(72) Inventors: Ka-Wai WONG, Palo Alto, CA (US); Xu CAO, Palo Alto, CA (US); Jie Lexi REN, Palo Alto, CA (US); Wai-cheong Daniel SO, Palo Alto, CA (US)

(21) Appl. No.: 17/880,952

(22) Filed: Aug. 4, 2022

Related U.S. Application Data
(60) Provisional application No. 63/231,072, filed on Aug. 9, 2021.

(51) Int. Cl. G01N 33/487; C12Q 1/6825

(52) U.S. Cl. CPC G01N 33/48721; C12Q 1/6825

(57) ABSTRACT

Detection of viral nucleic acids (NAs) and their variants is effected using nanopore technology. If the target wild type viral NA is single-stranded, it is mixed with its complementary NA, and also the unknown viral NA sample to be analyzed, followed by hybridization; while if the target wild type viral NA is double-stranded, it is mixed with the unknown viral NA sample only, then denatured and followed by hybridization. The hybridized products from either case are then subjected to translocation in the form of a translocation analysis, experiment or test through a nanopore device that measures the electrical signals induced through translocation events. The corresponding signal train is characteristic of an individual virus or variant and acts as a “fingerprint” facilitating rapid virus identification and discovery of a new variant.

### RELATED APPLICATION

The present patent application claims priority to Provisional Patent Application No. 63/231,072 filed Aug. 9, 2021, which is assigned to the assignee hereof and filed by the inventors hereof and which is incorporated by reference herein.

### BACKGROUND

### Technical Field

The present disclosure pertains to molecular diagnostics a system and a method for rapid detection of viral nucleic acid (NA) and its variant, and also discovery of a new variant using nanopore technology.

### Background Art

A virus is an infectious agent of sub-microscopic size that multiplies inside the living host cells. Its biological nature was first discovered from studies in 1892 by the Russian scientist Dmitry I. Ivanovsky and in 1898 by the Dutch scientist Martinus W. Beijerinck (Wagner, Robert R. and Krug, Robert M., “Virus”, Encyclopedia Britannica (https://www.britannica.com/science/virus)). The identification of the first reported human virus (Yellow Fever virus) dated back to 1900 (Oldstone, M. B. A., “History of Virology”, In Encyclopedia of Microbiology; Schmidt, T. M., Ed., Academic Press: Cambridge, Mass., 608-612, 2014). New species of human virus are being identified continuously every year, which now account for more than two-thirds of new human pathogens (Woolhouse, M.; Gaunt, E., “Ecological Origins of Novel Human Pathogens”, Crit. Rev. Microbiol. 33, 231-242, 2007).

Basically, a virus particle, namely a virion, is made of its genetic material, that is nucleic acid (NA) housed inside a protein shell, or capsid. As is the case with all other living organisms, its genetic material defines its identity. As such, diagnoses based on NA tests (e.g., qPCR, sequencing) are among the most sensitive and accurate methods to confirm the presence of a particular virus in any circumstance. During a pandemic as exemplified by the invasion of SARS-CoV-2 beginning in 2019, a rapid but accurate viral detection method is most sought-after for timely deployment of early medical treatment, unambiguous tracing of viral footprint and effective containment of transmission.

On the other hand, viruses are constantly experiencing changes, namely mutations, to their genetic material; i.e., the viral NA, resulting in variants which may enhance the ability of a virus to replicate, transmit or evade our immune systems.

Viral mutations can occur naturally during viral replication, or upon exposure to external physical and chemical effects intentionally imposed or present in certain environmental settings. There are different types of mutations, all resulting in different changes in genetic sequence. There are basically five types of mutations in a single-molecular level, namely “deletion”, “insertion” and “substitution” with regards to the change involving a single base. When a sequence or segment of bases are involved, a macro-level mutation occurs, which can be a “deletion”, “duplication”, “inversion”, “substitution” and “translocation”. FIGS. 1 and 2 show the basic single-molecular level and micro-level mutations in a single-stranded NA for illustration.

Briefly for single-molecular level mutations, “deletion” means one of the nucleic acid base (in a single-stranded entity) or base pair (in a double-stranded entity) is missing from the original sequence. “Insertion” means the incorporation of one nucleic acid base or base pair; while “substitution” means one of the nucleic acid base or base pair is replaced by another one.

When a short sequence of a NA is involved in changes, macro-level mutations occur. “Deletion” then means a sequence of nucleic acid bases (in a single-stranded entity) or base pairs (in a double-stranded entity) is missing from the original NA. “Duplication” means the repeated incorporation of the same short sequence of nucleic acid base or base pair following the original one. “Inversion” means the short sequence of nucleic acid base or base pair involved is reversed in a corresponding region. “Substitution” refers to the case when the short sequence of nucleic acid base or base pair involved is replaced by another short sequence with the same number of bases within. Finally, “translocation” represents the case when the short sequence of nucleic acid base or base pair involved moves to another region of the original NA.

The mutated virus; i.e., variants, can induce subsequent waves of mass infections with even stronger devastating effects. It is because a virus variant can severely weaken the performance of the existing molecular tests if the mutated region(s) overlap with the genetic target(s) adapted by the tests and thus the variants can easily evade the tests. False negative results then occur, and infection will carry on unnoticed. Nucleic acid sequencing technologies should be able to accurately detect emerging virus variants. It is noted, however, the associated turnaround time, analysis cost and technical complications have seriously limited their applicability.

As such, a novel fast screening technique to quickly identify new variants is of critical importance for a timely response to stop the variant from causing a new wave of mass infection.

Although the key subject of interest is on viral nucleic acids and their variants in the present context, the disclosed techniques can also be extended to application on NA sequence from any source; e.g., animal, plant, human.

### SUMMARY

Nanopore-based detection of viral nucleic acids (NAs) and their variants are provided by denaturing and annealing reactions between wild type and sample viral NAs, using a nanopore sensor and monitoring translocation of resulting hybridized products. The denaturing and annealing reactions is performed in a reaction compartment. The nanopore sensor uses nanopore material responsive to translocation events and the nanopore sensor provides electrical sensing of the nanopore material to provide a signal train indicating the translocation events. In the case of the target wild type viral NA being provided as a single-stranded wild type NA, a NA strand complementary to the wild type viral NA is prepared. For double-stranded NA, the mixture is denatured and annealed to provide hybridized products.

In one configuration, detection of viral nucleic acids (NAs) and their variants using nanopore material is performed by mixing wild type sample viral NAs, annealing the mixture to provide hybridized products, and subjecting the hybridized products to translocation detection and measurement by a nanopore-based device. Signal trains from different ones of the hybridized products are generated. The signal trains are analyzed to confirm the presence of the target wild type viral NA and known variants, and to detect the presence of an emergence variants with unknown sequences.

### BRIEF DESCRIPTION OF THE DRAWINGS

FIG. 1 is a diagram illustrating the three basic single-molecular level mutation in a single-stranded RNA.

FIG. 2 is a diagram illustrating the five basic micro-level mutation in a single-stranded RNA.

FIG. 3 is a diagram illustrating an exemplary process for the case of the target wild type NA and its variants being single-stranded.

FIG. 4 is a diagram illustrating an exemplary process for the case of the target wild type NA and its variants being single-stranded viral NA containing more than one type of NA variant.

FIG. 5 is a diagram illustrating an exemplary process for the case of the target wild type NA and its variants being double-stranded.

FIG. 6 is a diagram illustrating an application example of analyzing variants.

### DETAILED DESCRIPTION

### Overview

The present disclosure relates to a method and a system for rapid detection of a variant of a virus and discovery of a new variant even in a sample containing multiple suspected viruses and variants, with the use of nanopore technologies.

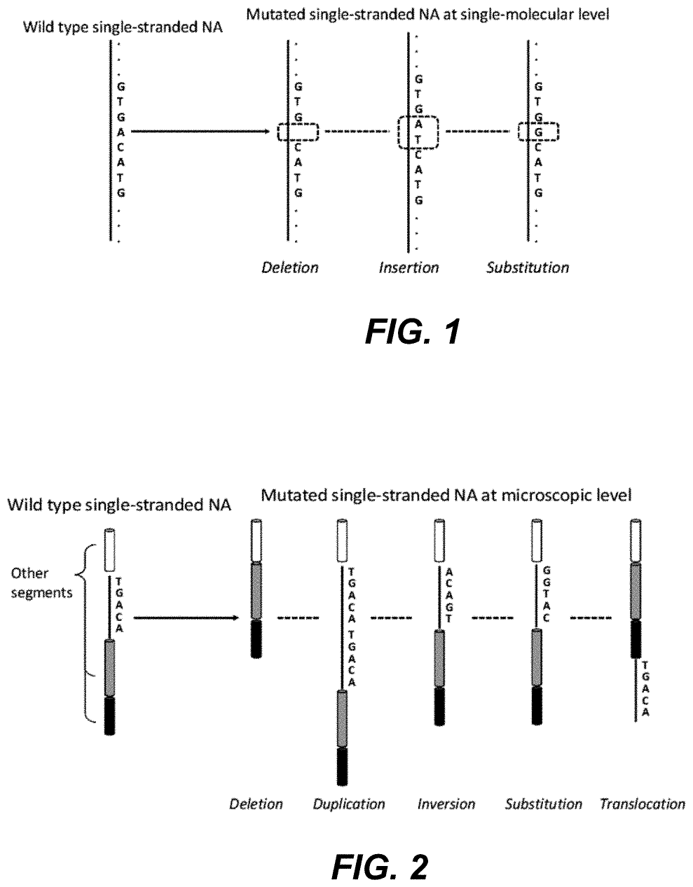

The disclosed techniques can be applied to both single-stranded viral NA or double-stranded viral NA. Although the essence and rationale for both cases are the same, the experimental or testing procedures are slightly different. As such, the detailed descriptions will be made separately for single-stranded and double-stranded viral NA as below. FIG. 1 is a diagram illustrating the three basic single-molecular level mutation in a single-stranded RNA. FIG. 2 is a diagram illustrating the five basic micro-level mutation in a single-stranded RNA.

The disclosed molecular diagnostic method and system based on nanopore technology, allows rapid detection of the virus, and also discovery of a new variant from the signal train measured from the translocation of viral nucleic acid (NA). The NA can be DNA or RNA or its derivative through a nanopore, which includes but is not limited to peptide NA (PNA) and any labeled or modified NA such as those fluorescence-labeled or biotinylated.

Nanopores can be broadly categorized into two types: biological and solid-state. Both types have been used and proven to be applicable for molecular detection. Biological nanopores, that are also known as transmembrane protein channels, are usually inserted into a planar substrate such as lipid bilayers, or liposomes to form the sensing platform. Examples are α-Hemolysin, MspA. Solid-state nanopores refer to those made of inorganic materials such as oxides (e.g., Al₂O₃, SiO₂), nitrides (e.g., Si₃N₄), 2D materials (e.g., graphene, MoS₂), polymers. Solid-state nanopores are usually fabricated by physical processes, for instance, ion or electron beam bombardment, electrochemical etching, ion-tracked etching.

The single-stranded wild type viral NA is hereafter named as “WT”. Although it is naturally a single-strand NA, it does also have its complementary NA, named as “cWT” hereafter, that can be prepared artificially by different molecular biological techniques, such as reverse transcription. To a mixture consisting of both WT and cWT, the unknown viral NA sample (namely, S) is added, which is suspected to be the wild type viral NA or its mutated form; i.e., its variant. Under appropriate annealing conditions, hybridized products of WT-cWT and S-cWT result. They will then be subjected to translocation analysis, experiment or test with nanopore and signal measurement. The term “wild type NA” as used herein references a viral NA which is being detected as a test sample to be identified based on the disclosed diagnostic method, as opposed to a known test sample. In that sense, “wild type NA” may in some cases be known, especially if the disclosed system is being tested. For purposes of this disclosure, “wild type NA” is the NA material under test, as opposed to the standard NA material against which the “wild type NA” is being tested. The intent is to be able to test an unknown sample as “wild type NA” but as with most test procedures, the test may also be performed on a known sample, for example to verify proper operation of the test equipment.

The measurement can be achieved by monitoring the variation of the blockage current induced by the translocation of hybridized product through an appropriately sized nanopore, which can be a bio-nanopore or a solid-state nanopore. Another method of measurement is achieved by monitoring the tunneling electrical signal (e.g., current, voltage, capacitance, and etc.) induced by the translocation of hybridized product through an appropriately sized nanopore, which can be a bio-nanopore or a solid-state nanopore, with suitable embedded electrodes able to initiate and detect the corresponding tunneling electrical signal.

If the sample viral NA is the wild type viral NA; i.e., S=WT, only one type of signal train can be observed. It is noted, however, if the sample viral NA is a variant of the wild type; i.e., S≠WT, a different signal train can be observed because the mutated region of the later will gives rise to a local structural variation (e.g., kink, loop, nick) in the hybridized product. Each local structural variation can lead to a unique signal feature whose position along the signal train truly reflects the position of the mutated point or region. Also, its signal profile can provide information about the type of structural variation, and help deduce the corresponding type of mutation. As such, a new variant can be identified quickly and easily, whose characteristic signal train can be recorded and stored in database for subsequent comparison and analysis.

On the other hand, if the wild type viral NA is double-stranded, consisting of a pair of complementary strands, hereafter named as “WT1” and “WT2”, and the sample viral NA, that is suspected to be the wild type viral NA or its variant, consisting of a pair of complementary strands, namely “S1” and “S2”, and “S1” is defined as one strand of the pair that is structurally close to “WT1”; while “S2” close to “WT2” in terms of base sequence. The wild type viral NAs and sample viral NAs are mixed, denatured and re-annealed, resulting in a mixture of four hybridized products: WT1-WT2, S1-S2, WT1-S2, WT2-S1. They will then be subjected to a translocation analysis, experiment or test with nanopore material, as in the case for single-stranded viral NA.

If the sample viral NA is the wild type viral NA; i.e., S1=WT1, and thus S2=WT2, only one type of signal train can be observed. On the other hand, if the sample viral NA is a variant of the original; i.e., S1≠WT1 and thus S2≠WT2, signal trains originating from four hybridized products: WT1-WT2, S1-S2, WT1-S2, WT2-S1 can be observed. Due to base complementary, WT1-WT2 and S1-S2 would give the same constant signal train; while WT1-S2 and WT2-S1 would give the same signal train with exactly the same spectral features. As such, two types of signal can be observed instead of four. As in the case for single-stranded viral NA analysis described supra, the local structural variations induced by the mutated region(s) can be reflected by unique signal feature(s) whose position(s) and profile along the signal train can confirm the presence of a variant.

Since each variant has its own form and mode of mutation, its signal train detected is characteristic of its own. Therefore, the present disclosure can also offer detection of multiple variants in a single sample in a single diagnostic test.

Since it is not required to identify an individual base and differentiate the exact base sequence of the viral NA as in well-known nanopore sequencing technologies which requires a complicated detection strategy, sophisticated post-measurement data treatment and related computation in order to achieve detection of a new variant, the present disclosure provides a unique method to screen viral samples and to detect emerging variants very rapidly.

A method and system enabling rapid viral identification and determination of a new variant is provided. Although viral nucleic acids and their variants are mainly discussed in the present context, the disclosed techniques can also be extended to application on an NA sequence from any source; e.g., animal, plant, human.

Detection of Single-Stranded Viral NA Wild Type and its Variant

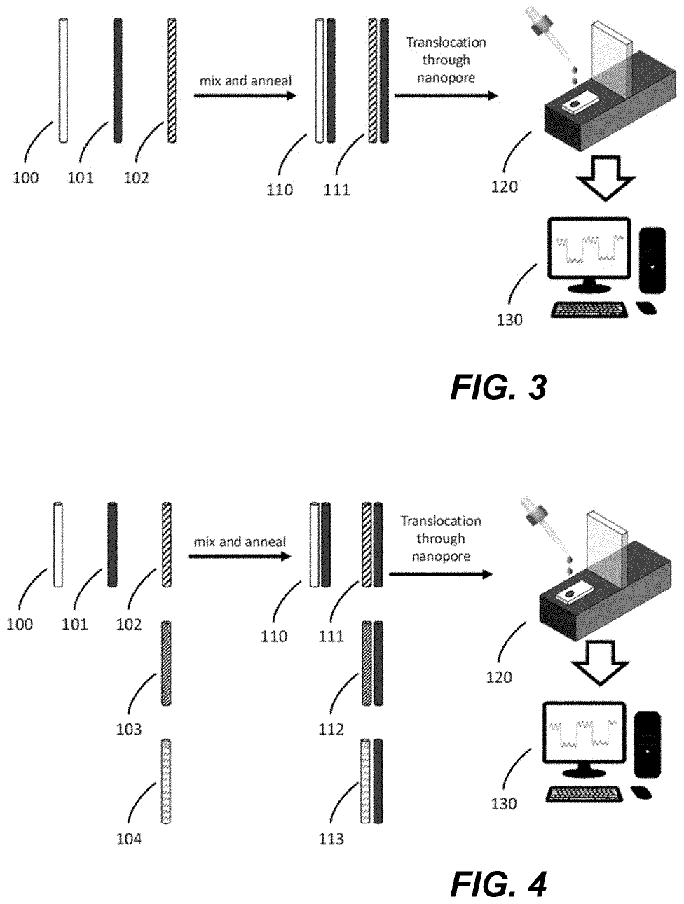

FIG. 3 is a diagram illustrating an exemplary process for the present disclosure if the target wild type NA and its variants are single-stranded. For a wild type single-stranded viral NA (WT) 100, its complementary NA (cWT) 101 is firstly synthesized using general molecular biological techniques; e.g., reverse transcription, and is mixed with WT.

To the mixture of WT and cWT, the sample viral NA (S) 102 is added. The resultant mixture is annealed to give two hybridized products: “WT-cWT” 110 and “S-cWT” 111. They are then subjected to a nanopore-based translocation analysis, experiment or test using a nanopore device 120. The nanopore device should further include a computer or other processor 130 to read and record the ion blockage current or other electrical and electronic signals from the built-in or embedded electrodes, and store the measurement data as the hybridized products pass through the nanopore material. The computer or other processor should also store the signal trains for hybridized products translocating the nanopore material. Those skilled in the art may use nanopore devices other than those described here, but also suitable for the disclosed technology.

If the signal trains arising from 110 and 111 are identical, it implies S=WT, and the presence of the target wild type viral NA in the sample is therefore confirmed. It is noted, however, if the sample viral NA is a variant of the wild type; i.e., S # WT, a different signal train can be observed because the mutated region of the later will gives rise to a local structural variation (e.g., kink, loop, nick) in the hybridized product. Each local structural variation can lead to a unique signal feature whose position along the signal train truly reflects the position of the mutated point or region. Also, its signal profile can provide information about the structural variation and thus help deduce the corresponding type of mutation. As such, a new variant can be identified, whose characteristic signal train is recorded and stored in database for subsequent comparison and analysis.

FIG. 4 is a diagram illustrating an exemplary process for the present disclosure if the single-stranded viral NA sample to be analyzed contains more than one type of NA variant. The disclosed techniques can also be applied to a sample containing more than one suspected sample viral NA. For a sample containing more than one suspected viral NA, for example three suspected viral NAs (102, 103, 104). After mixing and annealing, there will be four hybridized products 110, 111, 112, 113 resulting from hybridization of the wild type viral NA and the sample viral NAs with the complementary wild type viral NA; i.e., c-WT. As judged from the resultant signal trains, one can easily determine whether there is one or more variant, whose mutation characteristics (e.g., position, type) can also be revealed.

Detection of Double-Stranded Viral NA Wild Type and its Variant

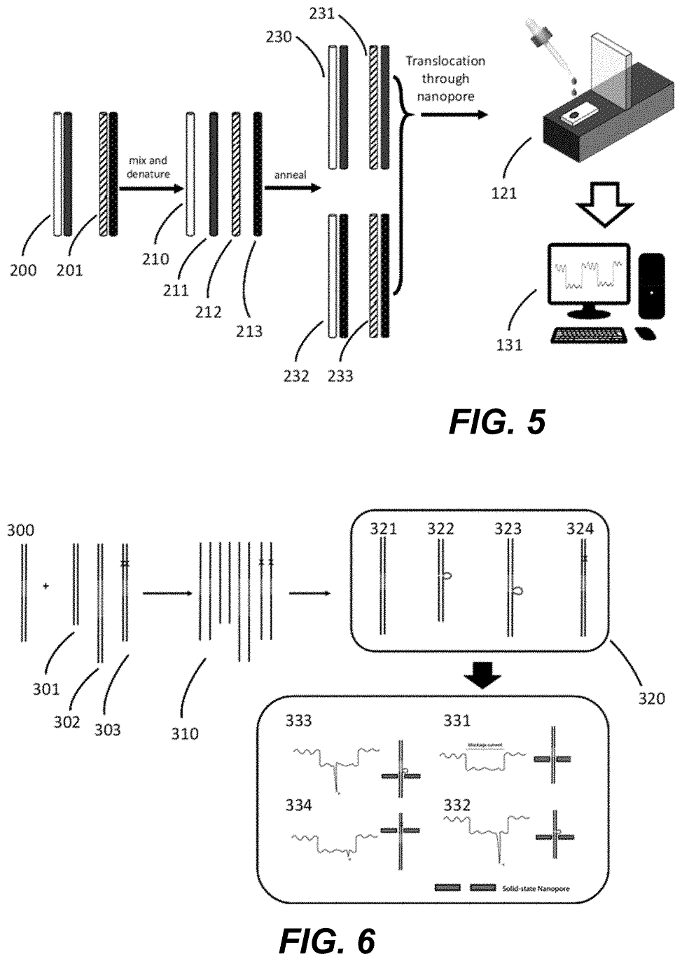

FIG. 5 is a diagram illustrating an exemplary process for the present disclosure if the target wild type NA and its variants are double-stranded. For a wild type viral NA that is a double-stranded NA, one more step is required. After mixing the wild type double-stranded viral NA 200 with the sample viral NA 201, a denaturing process is added before annealing to separate all the strands from each other, giving individual single strands 210, 211, 212 and 213. Afterwards, the whole mixture is annealed, giving four hybridized products 230, 231, 232, 233. If their signal trains are identical, it implies S1=WT1, and thus S2=WT2. The presence of the target wild type viral NA in the sample is therefore confirmed. If the sample viral NA is a variant of the wild type; i.e., S1≠WT1 and thus S2≠WT2, a different signal train can be observed because the mutated region of the later will gives rise to a local structural variation in the hybridized product, as in the case of detection of single-strand viral NA and its variant. Although there are four hybridized products: WT1-WT2 (230), S1-S2 (233), WT1-S2 (232), WT2-S1 (231), signal trains arising from WT1-WT2 and S1-S2 are identical, which is a constant signal train without any spike. Also, signal trains arising from WT1-S2 (232) and WT2-S1 (231) are the same as well due to base complementary. As such, two types of signal trains can be obtained instead of four. For the signal from WT1-S2 or WT2-S1, it should bear specific spike feature(s) originated from the local structural variation(s) whose position along the signal train truly reflects the position of the mutated point or region, and also the corresponding signal profile can provide information about the structural variation. These signal features help deduce the type of mutation. As such, a new variant can be identified, whose characteristic signal train is recorded and stored in a database for subsequent comparison and analysis.

Again, the disclosed techniques can also be applied to a sample consisting of multiple suspected double-stranded viral NAs, as in the case of detection of single-strand viral NA and its variant.

### Application Example

FIG. 6 is a diagram illustrating an application example for the present disclosure in analyzing variants. Here, a workable example of a diagnostic test on a sample containing 3 different variants of a wild type double-stranded viral NA 300 using the disclosed techniques is described. The type of mutations in the three variants are respectively “deletion” 301, “insertion” 302 and “point mutation” 303.

The sample with the three variants and the wild type are mixed and prepared in a solution with a concentration of 20 ng/μL with Taq 10x PCR buffer. A volume of 20 μL of the resultant solution is then subjected to the following annealing cycles to denature and then hybridize the strands into hybridized products:
(1) Stay at 95° C. for 10 minutes (Note: this is the step to denature all double-stranded NAs into single strands 310);
(2) Cool from 95° C. down to 85° C. at a cooling rate of −2° C./second;
(3) Stay at 85° C. for 1 minute;
(4) Cool from 85° C. down to 75° C. at a cooling rate of −0.3° C./second;
(5) Stay at 75° C. for 1 minute;
(6) Cool from 75° C. down to 65° C. at a cooling rate of −0.3° C./second;
(7) Stay at 65° C. for 1 minute;
(8) Cool from 65° C. down to 55° C. at a cooling rate of −0.3° C./second;
(9) Stay at 55° C. for 1 minute;
(10) Cool from 55° C. down to 45° C. at a cooling rate of −0.3° C./second;
(11) Stay at 45° C. for 1 minute;
(12) Cool from 45° C. down to 35° C. at a cooling rate of −0.3° C./second;
(13) Stay at 35° C. for 1 minute;
(14) Cool from 35° C. down to 25° C. at a cooling rate of −0.3° C./second;
(15) Stay at 25° C. for 1 minute;
(16) Cool from 25° C. down to 4° C. at a cooling rate of −0.3° C./second;
(17) Hold on 4° C. for 1 minute.

Afterwards, hybridized products 320 result, and four distinctive signal trains can be obtained corresponding to the products with strands from the wild type 321, and with strands from variants having “deletion” 322, “insertion” 333 and “point-mutation” 334.

As judged from the position and signal profile of any unique spectral feature along the signal train, the position of the mutated point or region and information about the structural variation induced can be obtained. These signal features help deduce the type of mutation. As such, a new variant can easily be identified, through comparison with existing known wild type virus and variants.

### CLOSING STATEMENT

The foregoing description is illustrative of particular embodiments, but it is not meant to be a limitation upon the practice thereof. The following claims, including all equivalents thereof, are intended to define the scope of the disclosed technology.

It will be understood that many additional changes in the details, materials, steps and arrangement of parts, which have been herein described and illustrated to explain the nature of the subject matter, may be made by those skilled in the art within the principle and scope of the invention as expressed in the appended claims.

What is claimed is:

1. A system for nanopore-based detection of viral nucleic acids (NAs) and their variants, the system comprising:
a reaction compartment providing denaturing and annealing reactions between wild type and sample viral NAs; and
at least one reaction compartment having nanopore material responsive to translocation events and a nanopore sensor providing electrical sensing of the nanopore material;
a monitoring device connected to the nanopore sensor, the monitoring device monitoring translocation of hybridized products through the nanopore sensor in the reaction compartment and indicates the corresponding signal train said translocation events.

2. The system of claim 1, wherein the at least one nanopore sensor detects and confirms both a wild type viral NA and variant with a known sequence, and also an emerging variant with an unknown sequence.

3. The system of claim 2, wherein the wild type viral NA and variant can be single-stranded or double-stranded.

4. The system of claim 1, wherein the at least one nanopore sensor probes and gives a signal train when a hybridized NA molecule translocates the nanopore material.

5. The system of claim 4, wherein at least one nanopore sensor consists of a nanopore material of any shape that allows translocation of a hybridized NA molecule.

6. The system of claim 5, wherein the nanopore material comprises a nanopore material comprising one of a group selected from a biological nanopore material, inorganic nanopore material, or organic nanopore material.

7. The system of claim 1, wherein the reaction compartment is directly connected to the at least one nanopore-based device or separated physically from the nanopore-based device.

8. The system of claim 1, wherein the reaction compartment is made of microfluidic or nanofluidic components, allowing denaturing and annealing of NAs held within.

9. A method of detection of viral nucleic acids (NAs) and their variants using nanopore material, comprising:
mixing wild type sample viral NAs;
annealing the mixture is annealed to provide hybridized products; and
subjecting the hybridized products to translocation detection and measurement by a nanopore-based device;
comparing signal trains from different ones of the hybridized products and analyzing the signal trains to confirm the presence of the target wild type viral NA and known variants, and to detect the presence of an emergence variants with unknown sequences.

10. The method of claim 9, further comprising:
in the case of a target wild type viral NA provided as a single-stranded, mixing the wild type, sample viral NAs and also the NA strand complementary to the wild type viral NA prepared using a molecular biological technique, then annealing the mixture to provide hybridized products, and
in the case of a target wild type viral NA provided as a double-stranded, mixing the wild type viral NA with the sample viral NAs, then denaturing and annealing the mixture to provide hybridized products.

11. The method of claim 9, further comprising:
providing, as a target wild type viral NA, a single-stranded NA; and
mixing the wild type, sample viral NAs and also the NA strand complementary to the wild type viral NA prepared using a molecular biological technique, then annealing the mixture to provide hybridized products.

12. The method of claim 9, further comprising:
providing, as a target wild type viral NA, a double-stranded NA; and
mixing the wild type viral NA with the sample viral NAs, then denaturing and annealing the mixture to provide hybridized products.

13. The method of claim 9, wherein the molecular biological technique comprises reverse transcription.

14. The method of claim 9, wherein the complementary NA comprises DNA, RNA, PNA or its derivatives.

15. The method of claim 9, wherein the complementary NA comprises DNA, RNA, PNA or its derivatives, labeled with a fluorescent tag or biotinylated tag.

16. The method of claim 9, wherein the translocation comprises monitoring and measuring translocation by blockage current measurement or tunneling electrical measurement induced by the translocation of the hybridized product through nanopore material having appropriately sized nanopores for the translocation detection and measurement.

17. The method of claim 9, further comprising:
controlling the guiding of a NA molecule to the nanopores of the nanopore material by microfluidic techniques or by electrokinetic techniques.

18. The method of claim 9, further comprising:
mixing a sample under test containing DNS strands with a solution as a test sample; and
processing the test sample through a plurality of annealing cycles, thereby hybridizing the strands, the annealing cycles comprising maintaining the test sample for plurality of cycles of maintaining a temperature of the test sample for a predetermined time period, followed by cooling the test sample to subsequent predetermined lower temperatures.
